(19) United States
(12) Patent Application Publication
(10) Pub. No.: US 2022/0327905 A1
(43) Pub. Date: Oct. 13, 2022

(54) ANALYSIS AND WARNING SYSTEM, METHOD AND DEVICE PREDICTING AN ONSET OF SLEEP

(71) Applicant: Leigh m. Rothschild, Miami, FL (US)

(72) Inventor: Leigh m. Rothschild, Miami, FL (US)

(21) Appl. No.: 16/863,563

(22) Filed: Apr. 30, 2020

(51) Int. Cl. G08B 21/06; A61B 5/00; A61B 5/0205; A61B 5/0531; A61B 5/1455

(52) U.S. Cl. CPC G08B 21/06; A61B 5/7275; A61B 5/4806; A61B 5/746; A61B 5/681; A61B 5/0205; A61B 5/0531; A61B 5/14551; A61B 5/0022; A61B 5/7405; A61B 5/742; A61B 5/7455; G06V 40/16

(57) ABSTRACT

Embodiments of the disclosure provide a system for predicting onset of sleep. The system includes a user worn device that includes a plurality of primary sensors to collect a plurality of primary data of a user, a first processor, in communication with the primary sensors, to receive the data and further process the data, wherein the processor determines an onset of sleep data in the user, a first communications module, in communication with the processor, transmits the onset of sleep data, a secondary device, that collects secondary data of the user, a secondary processor, that receives the onset sleep data from the first communications module and secondary data from the at least one secondary sensor, and combines the received onset sleep data and the secondary data to determine whether an onset of sleep event is possible and warn the user about the onset of sleep event.

### FIELD OF INVENTION

The present invention relates to a warning system more particularly, the present invention relates to an analysis and warning system, device and method for predicting an onset of sleep in a user.

### BACKGROUND OF THE INVENTION

Across the world humans falling asleep when inappropriate is a substantial problem. According to the National Sleep Foundation, about half of U.S. adult drivers admit to consistently getting behind the wheel while feeling drowsy. About 20% admit to falling asleep behind the wheel at some point in the past year—with more than 40% admitting this has happened at least once in their driving careers.

These figures show how prevalent drowsy driving is. This drowsy driving puts themselves and others at high risk.

Not only is Unintended Sleep (USL) a problem for drivers but a problem for students in the classroom and workers in a work environment.

Currently, several companies have mobile products that measure sleep patterns once the user falls asleep, but these devices do not predict the onset of sleep.

For drowsy driving prevention there are devices that can be placed inside a vehicle or worn on the fingers or ear that attempt to warn the users about USL. However, these devices are not convenient or practical to use in everyday situations.

Currently, one of the largest consumer items is the digital smart watch. It is clearly apparent that many consumers have adopted the smart watch as a useful/practical everyday device. Yet, none of these smart watches incorporates technology to detect USL.

### BRIEF DESCRIPTION OF DRAWINGS

The following detailed description of various embodiments is better understood when read in conjunction with the drawings provided herein. For illustrative purposes, exemplary embodiments are shown in the drawings; however, the presently disclosed subject matter is not limited to the specific systems and methods shown in those embodiments

FIG. 1 is a block diagram of a system for predicting onset of sleep, in accordance with an embodiment of the invention.

FIG. 2 is a block diagram of a device for predicting onset of sleep, in accordance with an embodiment of the invention.

FIG. 3 is a flow chart depicting a method for predicting onset of sleep, in accordance with an embodiment of the invention.

### DETAILED DESCRIPTION OF INVENTION

The present invention provides a solution for recording the usage of a firearm and helping a user during usage of the firearm using multiple sensors to gather various information.

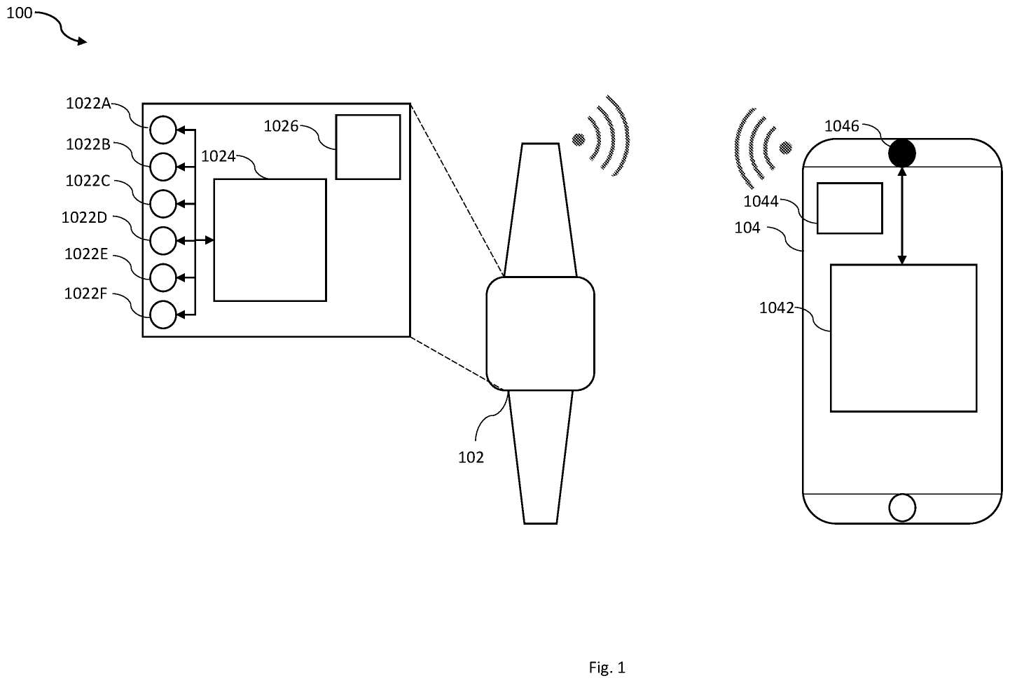

Referring to FIG. 1, a block diagram of a system 100 for predicting onset of sleep, in accordance with an embodiment of the invention. The system 100 includes a user worn device 102 and a secondary device 104.

According to an embodiment of the invention, the user worn device 102 may be anyone of a fitness band, a smart watch, and a smart ring.

According to yet another embodiment of the invention, the secondary device 104 is a smartphone.

The user worn device 102 includes a plurality of primary sensors 1022A, 1022B, 1022C, 1022D, 1022E, and 1022F (cumulatively referred to as primary sensors 1022 hereinafter). The user worn device 102 includes a first processor 1024, and a first communication module 1026.

According to an embodiment of the invention, the first processor 1024 is communicably connected to the sensors 1022 and the first communication module 1026. The primary sensors 1022 are configured to collect a plurality of primary data of a user. The primary sensors 1022 may be anyone or a combination of an accelerometer, an spo2 sensor, a hear rate sensor, a pulse rate sensor, and an electrodermal sensor. According to another embodiment of the invention, the plurality of primary data includes anyone or a combination of hypnagonic jerk , blood oxygen levels, pulse rate, heart rate, and electrodermal activity.

The secondary device 104 includes a second processor 1042 and a second communications module 1044. The secondary device 104 includes a secondary sensor 1046. The secondary sensor 1046 may be a CCD (charged couple) imaging sensor. The secondary sensor 1046 is configured to collect a secondary data of the user. According to an embodiment of the invention, the secondary data of the user may be anyone or a combination of facial data, head alignment data, and eye pattern data.

The user worn device 102 and the secondary device 104 may be communicably connected through a network. The network may be a short range or long range wireless network. The wireless network may be any one of a Wi-Fi, Bluetooth, cellular network.

The user worn device 102 may also include a warning module (not shown in the figure). The warning module may be anyone of a vibration module, an audio module, or an audio-visual module or a combination thereof.

According to an exemplary working embodiment, the primary sensors 1022 are connected to the first processor 1024. The first processor 1024 is connected to the first communications module 1026. The first communications module 1026, may be linked to secondary device 104. The secondary sensor 1046 may be located on the secondary device 104.

The first processor 1024 of the user device 102 will process all of the primary data of the user from the primary sensors 1022. The primary data of the user may then be sent to the secondary processor 1042. The transmission of primary data may occur from the first communications module 1026 to the second communications device 1044, which then gets transmitted to the second processor 1042. The second processor 1042 combines the primary data and the secondary data of the user to determine an onset of sleep. If the second processor 1042 determines an occurrence of an unintended onset of sleep (USL) the user of the user worn device 102 is continually warned with either vibrations sent through the user device 102 to the user and/or an audio warning. These Warnings will occur until the second processor determines that USL is not occurring.

The user worn device 102 will rely on a combination of the primary sensors 1022 and the secondary sensor 1046 to determine the onset of USL. By example one of the symptoms utilized in predicting USL is a hypnagogic jerk. This could be detected solely by the accelerometer; however, this action alone may not be totally predictive of USL. However, when combined with SPO2 data and other sensor data the software algorithms in the user worn device 102 can more accurately predict USL and accurately warn the user.

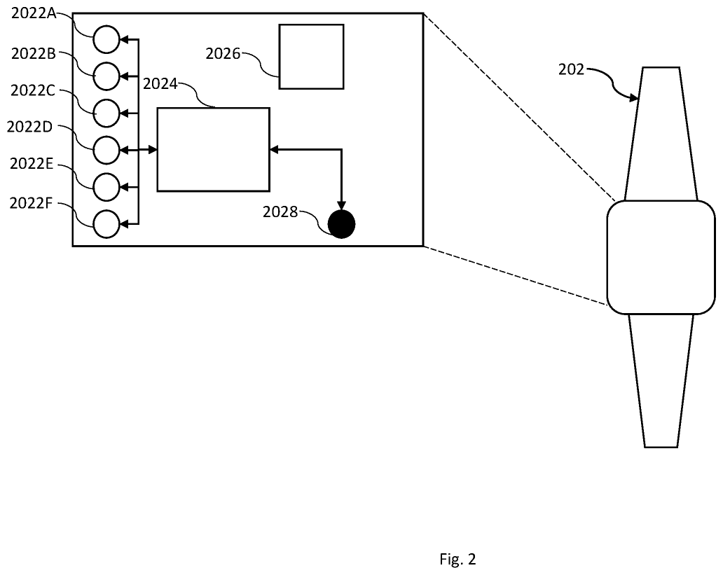

Referring now to FIG. 2, illustrating a block diagram of a device 200 for predicting onset of sleep, in accordance with an embodiment of the invention. The device 200 may be a user worn device particularly on wrist of the user. According to an embodiment of the invention, the user worn device 202 may be anyone of a fitness band, a smart watch, and a smart ring.

The user worn device 202 includes a plurality of primary sensors 2022A, 2022B, 2022C, 2022D, 2022E, and 2022F (cumulatively referred to as primary sensors 2022 hereinafter). The user worn device 202 includes a first processor 2024, a first communications module 2026, and a secondary sensor 2028.

According to an embodiment of the invention, the first processor 2024 is communicably connected to the primary sensors 2022 and the first communication module 2026. The primary sensors 2022 are configured to collect a plurality of primary data of a user. The primary sensors 2022 may be anyone or a combination of an accelerometer, an spo2 sensor, a hear rate sensor, a pulse rate sensor, and an electrodermal sensor. According to another embodiment of the invention, the plurality of primary data includes anyone or a combination of hypnagonic jerk , blood oxygen levels, pulse rate, heart rate, and electrodermal activity. The user worn device 202 may further a secondary sensor 2028. The secondary sensor 2028 may be a CCD (charged couple) imaging sensor. The secondary sensor 2028 is configured to collect a secondary data of the user. according to an embodiment of the invention, the secondary data of the user may be anyone or a combination of facial data, head alignment data, and eye pattern data.

The user worn device 202 may also include a warning module (not shown in the figure).

The warning module may be anyone of a vibration module, an audio module, or an audio-visual module or a combination thereof.

According to an exemplary working embodiment, the primary sensors 2022 are connected to the first processor 2024. The first processor 2024 is connected to the first communications module 2026. Also, the secondary sensor 2026 may be located on the user worn device 202.

The first processor 2024 of the user worn device 202 will process all of the primary data of the user from the primary sensors 2022. The secondary data of the user, collected using the secondary sensor 2028, may also be collected by the first processor 2024. The first processor 2024 combines the primary data and the secondary data of the user to determine an onset of sleep. If the first processor 2024 determines an occurrence of an unintended onset of sleep (USL), the user of the user worn device 202 is continually warned with either vibrations sent through the user device 102 to the user and/or an audio warning. These Warnings will occur until the first processor 2024 determines that USL is not occurring.

The user worn device 202 will rely on a combination of the primary sensors 2022 and the secondary sensor 2028 to determine the onset of USL. By example one of the symptoms utilized in predicting USL is a hypnagogic jerk. This could be detected solely by the accelerometer; however, this action alone may not be totally predictive of USL. However, when combined with SPO2 data and other sensor data the software algorithms in the user worn device 102 can more accurately predict USL and accurately warn the user.

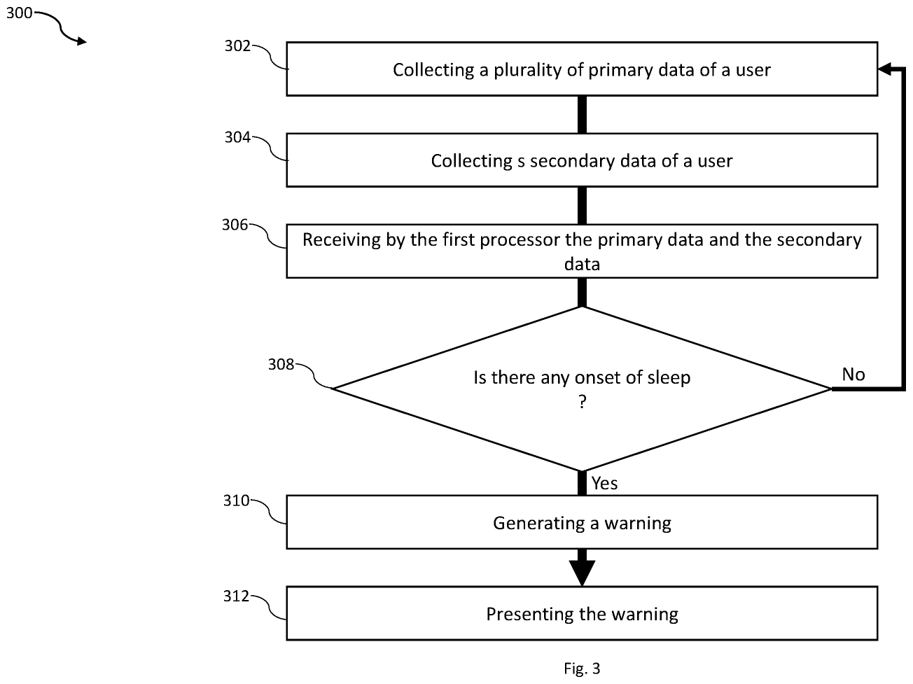

Referring now to FIG. 3, illustrating a flow diagram of a method 300 for predicting onset of sleep, in accordance with an embodiment of the invention.

At step 302, the plurality of primary data of the user (as described above in the description) is collected. The primary data collected may include anyone or a combination of hypnagonic jerk, blood oxygen levels, pulse rate, heart rate, and electrodermal activity.

At step 304, the secondary data of the user is collected. According to an embodiment of the invention, the secondary data of the user may be anyone or a combination of facial data, head alignment data, and eye pattern data.

At step 306, the first processor 1024 receives the primary data and the secondary data of the user. At step 308, the first processor 1024, determines whether there is any onset of unintended sleep (USL) or not. If no, then the method 300 reverts back to step 302. However, in case there is an onset of the unintended sleep then the method 300 moves to step 310 wherein a suitable warning is generated by the first processor 1024 for the user. The warning may be varied based on the level of the USL as can be determined by the first processor 1024. At step 312, the warning is presented to the user. The warnings may be any one or a combination of vibrations, and audio.

It is to be noted the processing from primary the sensors 1022 described herein may occur within the user worn device 102 or within the secondary device 104 linked to the user worn device 102, or in a separate computer server located at a distance from the user worn device 102 and secondary device 104 but communicating with the user worn device 102 and secondary device 104.

While certain embodiments have been described, these embodiments have been presented by way of example only, and are not intended to limit the scope of the present disclosure. Indeed, the novel methods, devices, and systems described herein may be embodied in a variety of other forms. Furthermore, various omissions, substitutions, and changes in the form of the methods, devices, and systems described herein may be made without departing from the spirit of the present disclosure. The accompanying claims and their equivalents are intended to cover such forms or modifications as would fall within the scope and spirit of the present disclosure.

What is claimed is:

1. A system for predicting onset of sleep comprising:
a user worn device comprising;
a plurality of primary sensors, configured to collect a plurality of primary data of a user;
a first processor, in communication with the plurality of primary sensors, configured to receive the plurality of data and further process the plurality of data, wherein the processor is further configured to determine an onset of sleep data in the user;
a first communications module, in communication with the processor, configured to transmit the onset of sleep data;
a secondary device, communicatively coupled to the user worn device;
at least one secondary sensor, configured to collect secondary data of the user;
a secondary processor, configured to perform the steps of;
receiving the onset sleep data from the first communications module;
receiving the secondary data from the at least one secondary sensor;
combining the received onset sleep data and the secondary data;
determine whether an onset of sleep event is possible; and
warning the user about the onset of sleep event to avert the sleep event.

2. The system of claim 1, wherein the user worn device is anyone of a fitness band, a smart watch, and a smart ring.

3. The system of claim 1, wherein the plurality of sensors include any one or a combination of an accelerometer, a spo2 sensor, a hear rate sensor, a pulse rate sensor, and an electrodermal sensor.

4. The system of claim 1, wherein the secondary sensor is a CCD imaging sensor.

5. The system of claim 1, wherein the plurality of primary data includes anyone or a combination of hypnagonic jerk , blood oxygen levels, pulse rate, heart rate, and electrodermal activity.

6. The system of claim 1, wherein the secondary data includes anyone or a combination of facial data, head alignment data, and eye pattern data.

7. The system of claim 1, wherein the secondary device is a smart phone connected to the user worn device through a network.

8. The system of claim 7, wherein the network is a wireless network like Wi-Fi, Bluetooth, cellular network.

9. The system of claim 1, wherein the user worn device further comprises a warning module.

10. The system of claim 9, wherein warning module is anyone or a combination of a vibration module, an audio module, or an audio-visual module.

11. The system of claim 1, wherein the secondary device further comprises a warning module.

12. The system of claim 11, wherein warning module is anyone or a combination of a vibration module, an audio module, or an audio-visual module.

13. The system of claim 1, wherein the secondary device is a remote computing device connected to the user worn device through a network.

14. The system of claim 7, wherein the network is a wireless network like Wi-Fi, or cellular network.

15. A user worn device for predicting onset of sleep comprising:
a plurality of primary sensors, configured to collect a plurality of primary data of a user;
a first processor, in communication with the plurality of primary sensors, configured to receive the plurality of data and further process the plurality of data, wherein the processor is further configured to determine an onset of sleep data in the user;
a first communications module, in communication with the first processor, configured to transmit the onset of sleep data;
at least one secondary sensor, configured to collect secondary data of the user;
wherein the first processor is configured to perform the steps of;
receiving the onset sleep data from the first communications module;
receiving the secondary data from the at least one secondary sensor;
combining the received onset sleep data and the secondary data;
determine whether an onset of sleep event is possible; and warning the user about the onset of sleep event to avert the sleep event.

16. The user worn device of claim 15, is anyone of a fitness band, a smart watch, and a smart ring.

17. The user worn device of claim 15, wherein the plurality of sensors include any one or a combination of an accelerometer, a spo2 sensor, a hear rate sensor, a pulse rate sensor, and an electrodermal sensor.

18. The user worn device of claim 15, wherein the secondary sensor is a CCD imaging sensor.

19. The user worn device of claim 15, wherein the plurality of primary data includes anyone or a combination of hypnagonic jerk , blood oxygen levels, pulse rate, heart rate, and electrodermal activity.

20. The user worn device of claim 15, wherein the secondary data includes anyone or a combination of facial data, head alignment data, and eye pattern data.
